(19) United States
(12) Patent Application Publication
(10) Pub. No.: US 2007/0068536 A1
(43) Pub. Date: Mar. 29, 2007

(54) Surgical incision protection device

(76) Inventor: Mark V. Rawski, Winfield, IL (US)

Correspondence Address: JANSSON SHUPE & MUNGER LTD., 245 MAIN STREET, RACINE, WI, 53403, US

(21) Appl. No.: 11/238,688

(22) Filed: Sep. 29, 2005

(51) Int. Cl. A61F 13/00

(52) U.S. Cl. 128/888; 128/846

(57) ABSTRACT

A surgical incision protection device has a body that has a longitudinal dimension and a lateral dimension; the a lateral length is less than the longitudinal length. The body has two ends. The body also has two arcuate surfaces. The arcuate distal surface is convex along the longitudinal dimension; the arcuate proximal surface is concave along the longitudinal dimension. The proximal surface radius of curvature is greater than the distal surface radius of curvature. The proximal surface also has a tunnel portion surface defining a distally-directed and laterally-extending cavity; the proximal surface has at least one skin-engaging portion surface.

### FIELD OF THE INVENTION

The invention relates to medical devices, particularly to surgical incision protection devices.

### BACKGROUND OF THE INVENTION

Abdominal-aspect prostrate surgery requires a vertical incision traversing the belt line of an average male. Post-operatively, clothing of a patient will frequently provide pressure to or rub against the incision. Such external interference results in discomfort to the patient, creates inflammation potentially inhibiting proper healing and provides means for infection.

To address the concept of incision protection, various strategies have been employed. One such strategy is that of Horn (U.S. Pat. No. 4,000,737). The rigidity of the Horn device decreases the range of motion of the individual. By running parallel to the incision the Horn device covers a large surface area of the body relative to the incision size. By providing small surface contact area relative to the force receiving portion of the Horn device, the proximally directed forces are translated to isolated parts of the body creating different areas of discomforture from the incision. Due to its bulk, the presence of the Horn device is apparent under normal clothing.

The wound protectors described by Purdy (U.S. Pat. No. 2,367,690) and Stevens (U.S. Pat. No. 3,026,874) enclose the wound controlling the environment and inhibiting free air flow. Moreover the Stevens and Purdy devices are bulky and rely on strap attachment means which are cumbersome. Further, do to their size and rigidity, they inhibit motion.

The devices of the prior art are enclosing, rigid, bulky, restrictive and in many instances cumbersome. A wound protector that would solve of the shortcomings of the prior art would be beneficial.

### OBJECTS OF THE INVENTION

It is an object of the invention to provide an improved incision protection device which overcomes some of the problems and shortcomings of the prior art, including those referred to above.

Another object of the invention is to provide a lightweight incision protection device.

Another object of the invention is to provide an incision protection device which is unobtrusive under clothing.

Still another object of the invention is to provide an incision protection device that distributes forces over a greater area of skin to increase comfort.

Yet another object of the invention is to provide an incision protection device that by being oriented transverse to the incision is not required to travel the entire length of the incision.

Another object of the invention is to provide an incision protection device allowing for free air flow.

Still another object of the invention is to provide an incision protection device that is easily modifiable to provide counter pressure to the incision at an appropriate time during the healing process.

Still another object of the invention is to provide an incision protection device that is easily modifiable to provide medication to the incision at an appropriate time during the healing process.

Still another object of the invention is to provide an incision protection device that is easily modifiable to provide thermal therapy to the incision at an appropriate time during the healing process.

Another object of the invention is to provide a incision protection device of a monolithic structure.

Another object of the invention is to provide a incision protection device which is formable.

Another object of the invention is to provide a incision protection device which is easy and economical to produce.

Another object of the invention is to provide a incision protection device which is flexible and comfortable.

Another object of the invention is to provide a incision protection device which can be easily secured with respect to the body in a variety of ways.

How these and other objects are accomplished will become apparent from the following descriptions and the drawings.

### SUMMARY OF THE INVENTION

The invention involves a surgical incision protection device. The surgical incision protection device has a body that has a longitudinal dimension and a lateral dimension; the lateral length is less than the longitudinal length. The body has two ends. The body also has two arcuate surfaces. The arcuate distal surface is convex along the longitudinal dimension; the arcuate proximal surface is concave along the longitudinal dimension. The proximal surface radius of curvature is greater than the distal surface radius of curvature. The inventor is using the phrase “radius of curvature” to allow him to more easily compare the degree of curvature or the various surfaces (e.g., the longitudinal cross-section of the body will result in a proximal surface profile that is closer to linear than the more arcuate distal surface profile); by use of this phrase, the inventor is not limiting these surfaces to circular profiles. The proximal surface also has a tunnel portion surface defining a distally-directed and laterally-extending cavity; the proximal surface has at least one skin-engaging portion surface.

It is preferable for the distal surface and the proximal surface to meet at the first end at a line. It is more preferable for the proximal surface to be configured for frictional engagement with human skin. Although the device's potential application is not limited to the torso of the human body, in certain applications it is even more preferable for the surgical incision protection device to have the proximal surface configured for circumferential installation about the waistline of a patient.

In some instances where the proximal surface is configured for engagement with human skin, it is advantageous to have an adhesive portion of the proximal surface that is coated with an adhesive.

Additionally, in some instances where the proximal surface is configured for engagement with human skin, it is advantageous for the proximal surface to be customizable to be complementary to shape vagaries of a patient. Patients come in various shapes in profile, in part dependant on the fitness of the patient. It is advantageous for the device to be customizable in a manner that conforms to the profile of the patient. Examples of customizability are thermoformability and moldability.

In certain applications, it is preferable for the body of the surgical incision protection device is to be monolithic.

It is preferable that the surgical incision protection device body is made of a flexible, non-crushable material. By the term “non-crushable”, inventor means that the material may be compressible and flexible, but that under pressure from a directional force, it will not significantly lose its shape. Such a material, for example, is a closed-cell polyethylene foam.

In certain other embodiments, there is a midline, which midline runs laterally along the proximal surface through a point that is longitudinally mid way between the first end and the second end. In certain of these embodiments, it is advantageous for the tunnel portion to be positioned along the midline. It is preferable for the proximal surface to be flexible along the midline. In this way, the concavity of the proximal surface may be selectively varied. This variation of concavity through flexing allows for rough customizing to the shape vagaries of the patient as a position-maintaining force is applied to the distal surface.

If the body is not monolithic, it is desirable for the tunnel portion to be semi-rigid whereby radial deformation of the cavity is resisted. This semi-rigidity may be accomplished through integrated molding during the fabrication process, or through the introduction of reinforcing strips (e.g., aluminum) to the interior surface of the tunnel.

It is a preferred embodiment to include a medicant receiving surface integral with the tunnel portion surface. The medicant receiving surface is configured and arranged to hold therapeutic materials in proximity to skin of a human. Examples of such medicant receiving surface is a common gauze pad, but may include a device as simple as the surface of a cold pack that is positioned such that the medicant-coated cold-pack surface is placed in contact with the skin through the application of force to the distal surface of the device. Examples of such medicants are antibiotics, fungistats, or other antiseptics, analgesics, coagulants, balms (such as aloe vera gel), or moisturizers.

In certain embodiments, it is preferable for the surgical incision protection device to include a fastener member attached to the distal surface. It is more preferable for the fastener member to be a mating surface of a hook and loop connection device. Hook and loop connection devices, such as those sold under the VELCRO® brand, have a hook portion strip surface and a complementary, mating loop portion strip surface. By the term “mating surface of a hook and loop connection device”, the inventor means a portion of a strip of either the hook or loop portion surface. In other embodiments, it is preferable for the fastener member to be a belt (including a strap or a conventional garment belt) attached to the distal surface. Attachment can be through any means including hook and loop interaction or frictional engagement.

Preferably, the device further comprises a thermal unit for applying thermal therapy (heat or cold) to the surgical incision. The thermal unit may be a reservoir of thermal material within the body capable of being heated or chilled. Such thermal material is frequently found in the health-care field, and specifically include the gel-filled hot/cold packs of flexible material (for example, the material used in ELASTO-GEL® Hot/Cold Wrap).

In another preferred embodiment, the surgical incision protection device of has a body that is of a material that is capable of maintaining a temperature.

It is another aspect of this invention to provide a surgical incision protection device that has a monolithic body. The body is of a flexible, non-crushable material that is capable of maintaining a temperature. The body has a longitudinal dimension of a longitudinal length and a lateral dimension of a lateral length. The lateral dimension is less than the longitudinal dimension. The body has first and second ends. The monolithic body further has an arcuate distal surface and an arcuate proximal surface. The arcuate distal surface has a first radius of curvature and is convex along the longitudinal dimension. The arcuate proximal surface has a second radius of curvature, which is concave along the longitudinal dimension. The said second radius of curvature is greater than the first radius of curvature. The proximal surface has a tunnel portion surface defining a distally-directed and laterally-extending cavity. The proximal surface also has a skin-engaging portion surface that is configured for circumferential installation about the waistline of a patient in engagement with the skin. The skin-engaging portion surface is customizable to be complementary to shape vagaries of the patient.

It is another aspect of this invention to provide a surgical incision protection device with a body that has a first and second laterally-tapered ends. The body also has an arcuate, non-slippery upper surface extending longitudinally between the first end and the second end. By the term “non-slippery upper surface”, the Inventor is referring to a surface that has a higher coefficient of static friction than that of polished polyvinyl chloride. One of the purposes of the non-slippery, frictionally-engaging surface is that of maintaining the device in place with respect to the skin of the patient through frictional engagement with the clothing of the patient. The body has a lower surface extending longitudinally between the first end and the second end. The lower surface has a tunnel portion surface and a skin-engaging portion surface. The tunnel portion surface defines a distally-directed, laterally-extending, non-deformable tunnel. The skin-engaging portion surface is configured for circumferential installation about the waistline of a patient in engagement with the skin of the patient.

Further details, advantages and features of this invention are given by the following description with reference to the enclosed drawings.

### BRIEF DESCRIPTION OF THE DRAWINGS

FIG. 1 is a perspective view of a preferred embodiment of the invention;

FIG. 2 is a top view the embodiment depicted in FIG. 1;

FIG. 3 is a elevational view of the proximal side of the embodiment depicted in FIG. 1;

FIG. 4 is a top elevational view of the embodiment depicted in FIG. 1 showing a range of motion;

FIG. 5 is a side view of a patient showing the end view of the device as it is installed;

FIG. 6 is a front view of a patient showing the distal surface of the device as it is installed on a patient;

FIG. 7 is a top view of the device installed on a patient;

FIG. 8 is a view of the proximal side of the device showing an optional securement means;

FIG. 9 is a view of the distal side of the device showing an alternate optional securement means;

FIG. 10 is a top view of the device showing an optional insert;

FIG. 11 is a top view of the embodiment depicted in FIG. 10 as it is installed on a patient;

FIG. 12 is a view of the proximal side showing an optional void;

FIG. 13 is a sectional view of the device of FIG. 12 taken along the 13-13;

FIG. 14 is a side view of a patient showing the end view of an alternative embodiment of the device as it is installed;

### DETAILED DESCRIPTION OF PREFERRED EMBODIMENTS

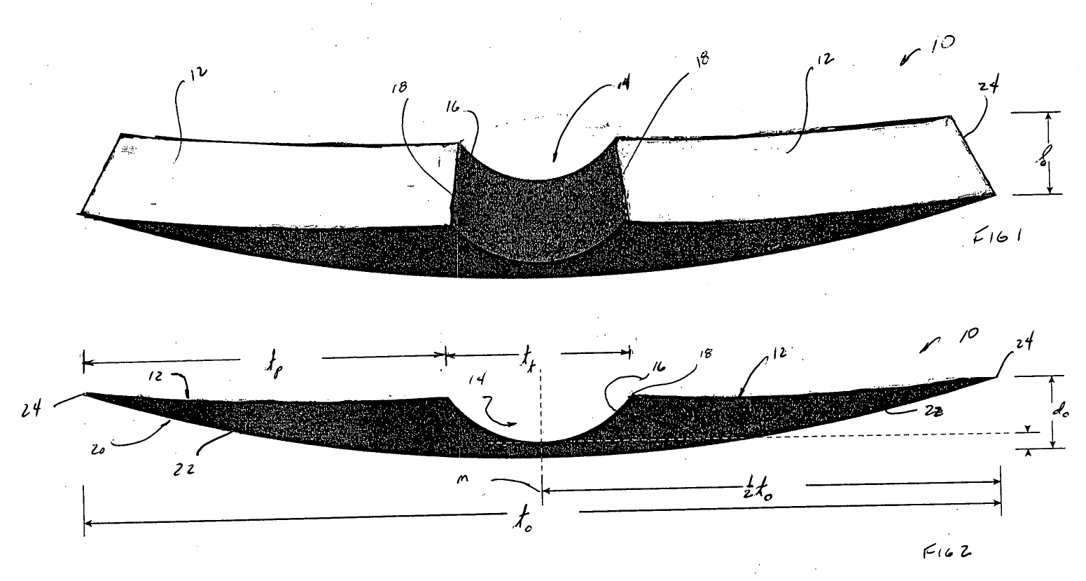

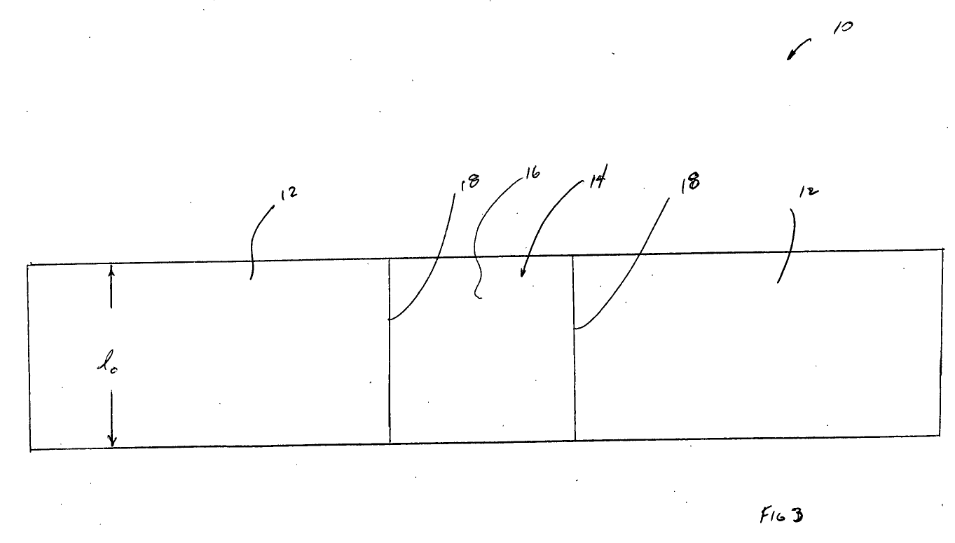

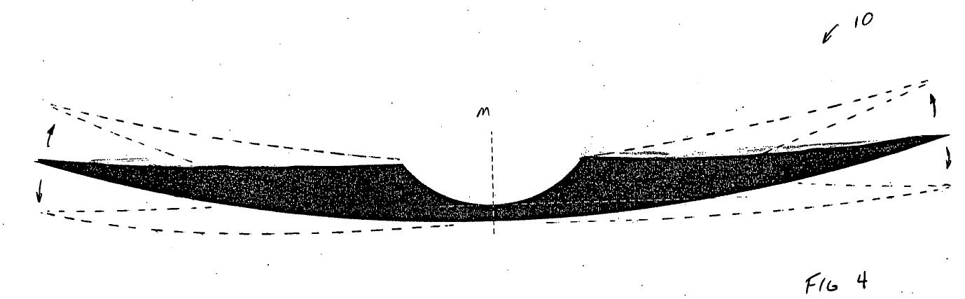

FIGS. 1 and 2 depict the device 10. Device 10 has proximal skin-engaging surfaces 12. As used in the description herein, the term “proximal” will refer to a direction nearer to the skin surface of a patient whereas “distal” will refer to a direction outward from the skin of a patient. It will be recognized that such terms “distal”, “proximal”, “longitudinal”, “lateral”, “top”, and “bottom”, are used for ease in describing the preferred embodiment; it will be further recognized that embodiments of the device can be used in various orientations such that the use directional terms in the description of the device may not be read to be limiting.

Device 10 has an overall longitudinal dimension tₒ, a lateral dimension lₒ, and a transverse depth dₒ.

As device 10 is intended for installation on a human patient about the waist, proximal skin-engaging surfaces 12 are arcuate in the longitudinal direction to accommodate the abdominal circumferential curvature of the human patient (best seen in FIG. 5). Along the longitudinal dimension tₒ, arcuate proximal skin-engaging surface 12 has a large radius of curvature resulting in a nearly planer surface in the longitudinal-lateral plane. Although the preferred embodiment illustrated does not include curvature along the lateral dimension of proximal skin-engaging surface 12, it is contemplated by the inventor that predetermined lateral curvature could create a more comfortable fit with certain patients.

Between the two proximal skin-engaging surfaces 12 is tunnel 14. Tunnel 14 is bounded by tunnel surface 16. Tunnel surface 16 meets proximal skin-engaging surface 12 at linear boundaries 18.

Device 10 has an arcuate distal surface 20. Arcuate distal surface 20 has a radius of curvature which is less than the radius of curvature of proximal skin-engaging surface 12. In this manner, a crescent, longitudinal cross-sectional shape (as best seen in FIG. 2) is obtained. With the crescent shape, tapered end wings 22 meet at end lines 24.

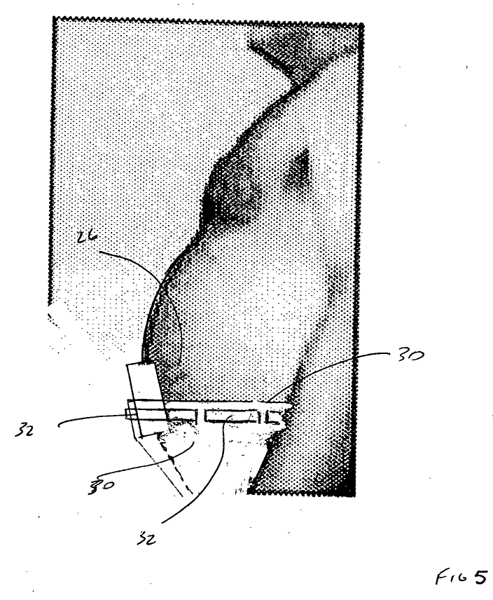

As seen in FIG. 4, the device 10 may be of a flexible material such that it can flex along a laterally-running mid-line m to assist in conformability about the torso of a patient.

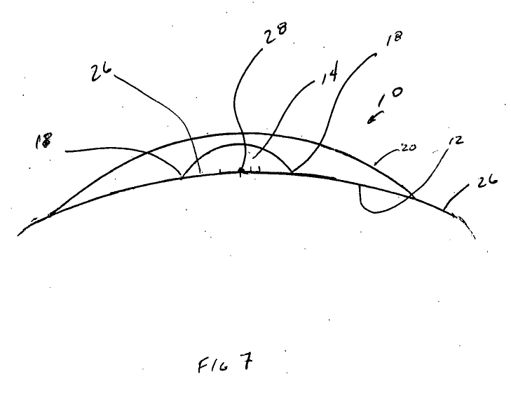

As seen in FIGS. 5, 6, and 7, the device is installed such that the proximal skin-engaging surface 12 is in contact with the skin 26 of the torso of a patient. Device 10 is placed such that tunnel 14 is positioned above incision 28 and linear boundaries 18 straddle incision 28. Tunnel 14 forms an arched barrier protecting incision 28 from external contact while allowing for air flow through tunnel 14 over incision 28.

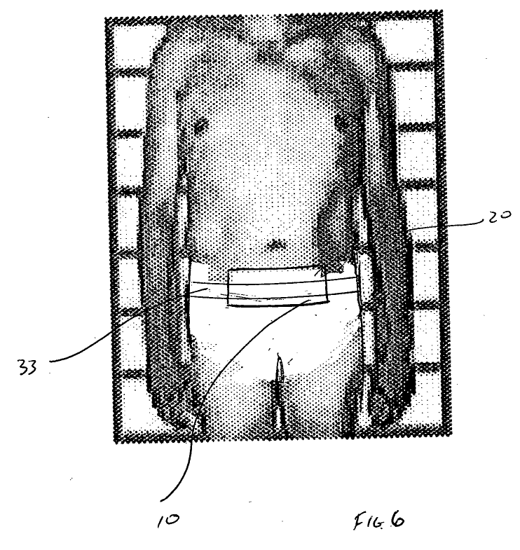

Device 10 may be held in position on the torso of a patient, straddling incision 28 by various means. As illustrated in FIG. 5, device 10 is positioned on skin 26 and is intermediate between skin 26 and the outer garments of the patients such as pants 30. Additional position-maintaining pressure may be brought to bear through the use of an ordinary belt 32 applied externally to pants 30. As belt 32 is cinched, a proximally-directed force is imparted through pants 30 to distal surface 20. As an alternative, as seen in FIG. 6, a strap 33 may be applied directly to distal surface 20 in a manner to apply position-maintaining pressure to device 10 to hold it in place on the patient. In such embodiments, the strap 33 will typically be hidden from view by the patient's clothes.

As illustrated in FIGS. 5 and 6, where pressure applied to the distal end is designed to maintain the position of the device 10 on a patient, it is desirable the skin-engaging surface 12 have a surface area (defined by the longitudinal tₚ and lateral lₒ dimensions) much greater than the surface area of the opening of tunnel 14 (which opening surface area is determined by longitudinal dimension tₜ by lateral dimension lₒ). Making use of the arch-bridge physics, proximally-directed, position-maintaining force applied to distal surface 20 is translated to wings 22 onto skin-engaging portion 12, thereby protecting incision 28.

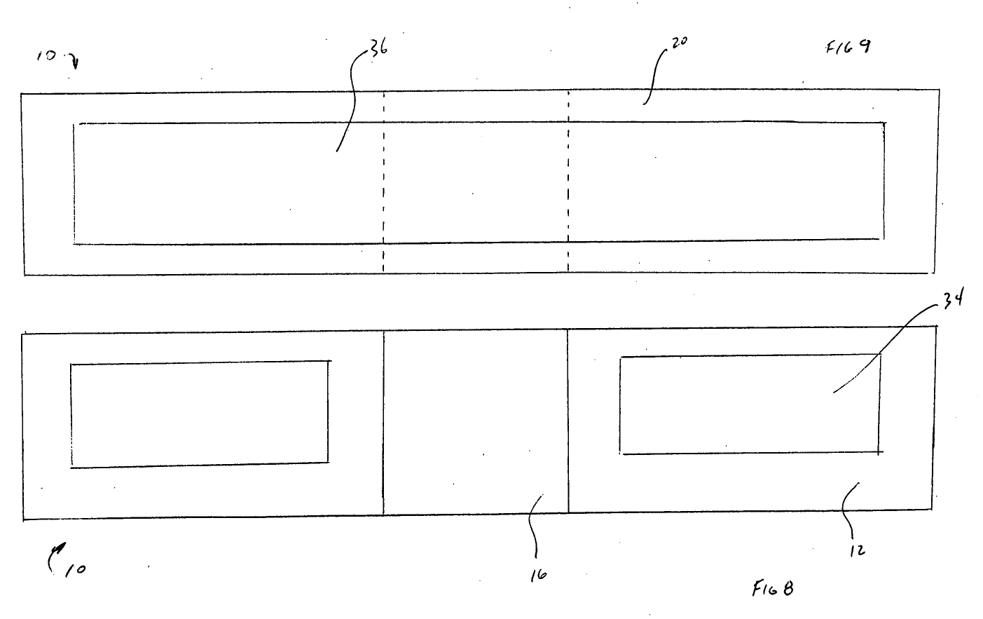

FIG. 8 illustrates another position-maintaining means. Adhesive 34 is applied to skin-engaging surface 12. In this manner, device 10 is positioned above skin 26 in such a manner that tunnel 14 is in protective proximity to incision 28. Hand pressure is then provided to distal surface 20 to urge adhesive-containing skin-engaging surface 12 into joining contact with skin 26.

Suitable adhesives are well known in the industry and include adhesives typically used for therapeutic adhesive strips, such as BAND-AID® adhesive strips. Such adhesive is of the type that is conducive for use with skin, easily removed without painful adhesion to the skin, of sufficient adhesive strength to maintain the device 10 in position while being resistant to dilution by water (e.g., during bathing).

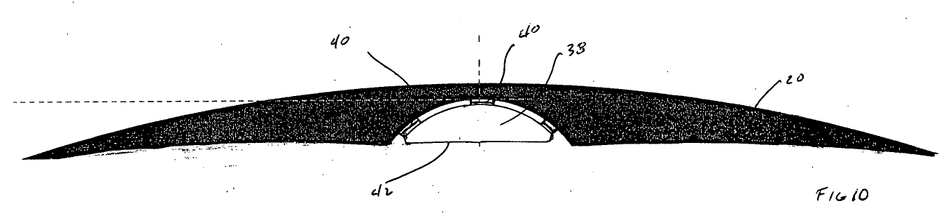

FIG. 9 illustrates yet another means of maintaining the position of device 10 with respect to skin 26 of a patient. Device 10 is maintained in position through a hook and loop connection as found in VELCRO® connecting strips. Hook and loop arrangements have two cooperating surfaces, a hook surface and a loop surface. In use, a hook surface is attached to a first part to be joined while a loop surface is attached to the second part to be secured with respect to the first part. Hook-surface strip 36 is attached to distal surface 20. Hooks of hook-surface strip 36 will interact with some outer garment materials (e.g., felt). In such cases where there is interaction between the hook-surface strip 36 and outer clothing, no further position-maintaining actions are necessary other than to position the device 10 on skin 26 and allow outer garment such as pants 30 to hold device 10 in place. Alternatively, with some tight fabrics, it may be necessary to affix the complementary loop-surface strip (not shown) on the proximal interior surface of the garment (e.g., through sewing or tack glue) to properly maintain the position of device 10 about incision 28.

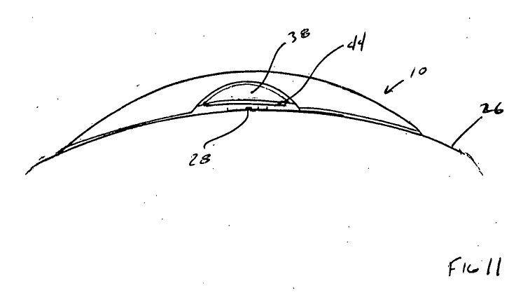

FIGS. 10 and 11 illustrate therapeutic applications beyond that of mere protection of the wound. As seen in FIG. 10, gel-filled sac 38 is affixed within tunnel 14 by means of complementary VELCRO® strips 40. Gel-filled sac 38 is typically of the type that will hold temperature selectively applied to it (i.e., may be chilled or heated as is therapeutically desired). Gel-filled sac 38 may be dimensioned such that proximal sac surface 42 extends proximally inwardly from skin-engaging surface 12 such that gel-filled sac 38, when device 10 is properly installed on the skin 26, will apply counter-pressure to incision 28 to help reduce scaring of the incision.

Alternatively, gel-filled sac 38 may be dimensioned such that proximal sac surface 42 is distal from skin-engaging surface 12 when device 10 is installed on the skin 26. In such cases, thermal therapy may be applied without contact with the incision 28 (e.g., at a time when the wound is fresh). Moreover, when gel-filled sac 38 is dimensioned in such a manner as to cause its proximal sac surface 42 to be distal of the proximal skin-engaging surface 12, space is created between sac surface 42 and skin 26 to allow for applications of medication 44 as seen in FIG. 11 (such as salve).

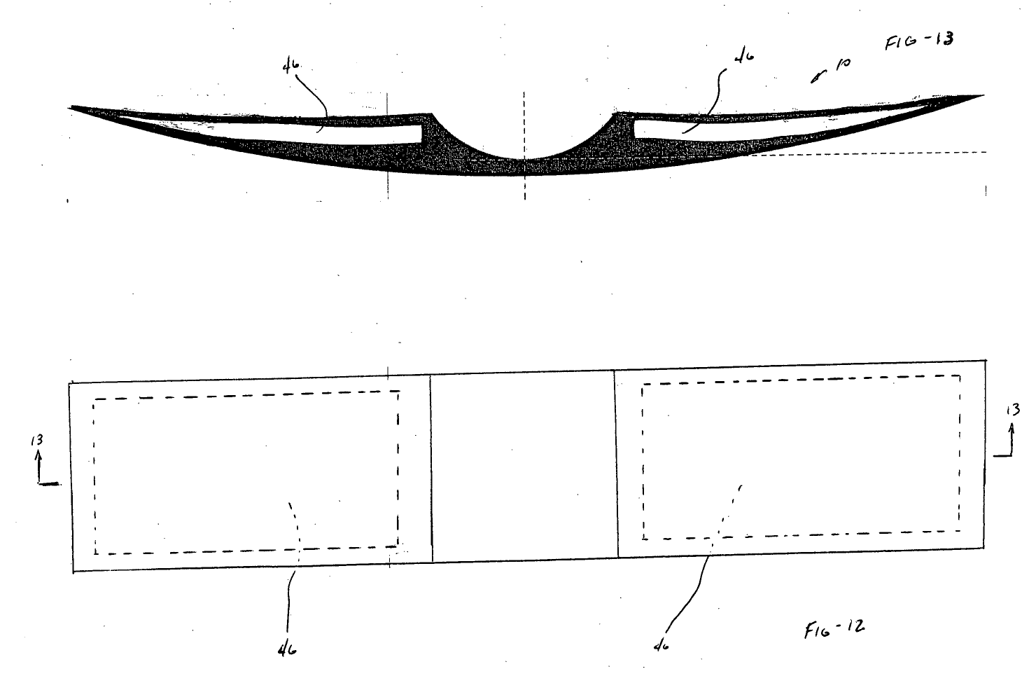

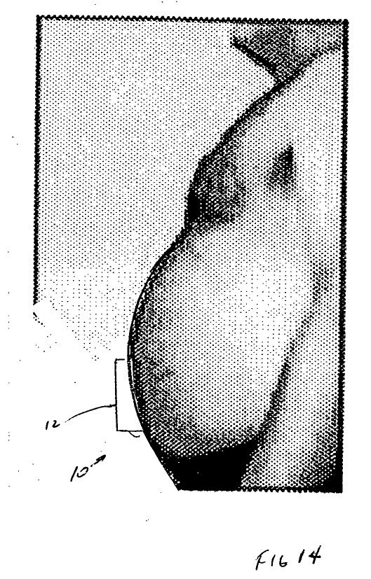

As seen in FIGS. 12 and 13, device 10 may further include integral gel reservoirs 46 which allows entire device 10 to be chilled or heated as is therapeutically desired.

It is preferable that device 10 be made of a material that is light-weight. Moreover, for purposes of cleanliness of the wound, a closed cell foam is desirable. Such suitable closed-cell foam is closed-cell polyethylene.

Alternatively, device 10 may be composed entirely of a chillable material. In this way, device 10 may be chilled prior to installation to allow for the comfort of the patient.

Regardless, it is imperative that tunnel 14 be non-crushable. To assist in maintaining the shape of tunnel 14, reinforcement, such as aluminum strips, may be installed longitudinally along tunnel surface 16 in a manner to protect against proximal deformation of tunnel 14.

Alternatively, to provide additional comfort for a patient, device 10 may be made of a conformable material such as visco-elastic memory foam.

Device 10 may also be made of a custom-moldable rubber material such as ethylene vinyl acetates (which include ELVAX 250, available from DuPont Company, Wilmington, Del., that has 28% vinyl acetate; ESCORENE available from Exxon Corporation; and ULTRATHENE from Quantum Company). As illustrated in FIG. 14, such custom-moldability is particularly advantageous for patients with broad contours. Custom-moldability in such circumstances allows for distal surface 20 to maintain a more vertical orientation thereby reducing potential rotation due to application of position-maintaining force induced by strap 37 or belt 38.

While the principles of the invention have been shown and described in connection with but a few embodiments, it is to be understood that such embodiments are by way of example and are not limiting.

I claim:

1. A surgical incision protection device comprising:
a body having a longitudinal dimension of a longitudinal length, a lateral dimension of a lateral length less than the longitudinal length, a first end, and a second end, the body further having,
an arcuate distal surface of a first radius of curvature, convex along the longitudinal dimension;
an arcuate proximal surface of a second radius of curvature, concave along the longitudinal dimension, said second radius of curvature greater than the first radius of curvature; said proximal surface with:
a tunnel portion surface defining a distally-directed and laterally-extending cavity; and
at least one skin-engaging portion surface.

2. The surgical incision protection device of claim 1 wherein the distal surface and the proximal surface meet at a first line at the first end.

3. The surgical incision protection device of claim 2 wherein the proximal surface is configured for frictional engagement with human skin.

4. The surgical incision protection device of claim 1 wherein the proximal surface is configured for circumferential installation about the waistline of a patient.

5. The surgical incision protection device of claim 1 wherein an adhesive portion of the proximal surface is coated with an adhesive.

6. The surgical incision protection device of claim 1 wherein the proximal surface is customizable to be complementary to shape vagaries of a patient at a point of installation on the patient.

7. The surgical incision protection device of claim 1 wherein the body is monolithic.

8. The surgical incision protection device of claim 7 wherein the body is made of a flexible, non-crushable material.

9. The surgical incision protection device of claim 8 wherein the material is a closed-cell polyethylene foam.

10. The surgical incision protection device of claim 1 wherein the tunnel portion is positioned along a midline, said midline running laterally along the proximal surface at a point longitudinally mid way between the first end and the second end.

11. The surgical incision protection device of claim 10 wherein the proximal surface is flexible along the midline, whereby the concavity of the proximal surface may be selectively varied.

12. The surgical incision protection device of claim 10 wherein the tunnel portion is semi-rigid whereby radial deformation of the cavity is resisted.

13. The surgical incision protection device of claim 1 further comprising a medicant receiving surface integral with the tunnel portion surface, said medicant receiving surface configured and arranged to hold therapeutic materials in proximity to skin of the patient.

14. The surgical incision protection device of claim 1 further comprising a fastener member attached to the distal surface.

15. The surgical incision protection device of claim 14 wherein the fastener member is a mating surface of a hook and loop connection device.

16. The surgical incision protection device of claim 14 wherein the fastener member is a belt attached to the distal surface.

17. The surgical incision protection device of claim 1 device further comprising a thermal unit.

18. The surgical incision protection device of claim 1 wherein the body is of a material that is capable of maintaining a selected temperature.

19. A surgical incision protection device comprising:
a monolithic body of a flexible, non-crushable material that is capable of maintaining a temperature, having a longitudinal dimension of a longitudinal length, a lateral dimension of a lateral length less than the longitudinal length, a first end, and a second end, the monolithic body further having:
an arcuate distal surface of a first radius of curvature, convex along the longitudinal dimension;
an arcuate proximal surface of a second radius of curvature, concave along the longitudinal dimension, said second radius of curvature greater than the first radius of curvature; said proximal surface with:
a tunnel portion surface defining a distally-directed and laterally-extending cavity; and
a skin-engaging portion surface configured for circumferential installation about the waistline of a patient in engagement with the skin of the patient, customizable to be complementary to shape vagaries of the patient.

20. A surgical incision protection device with a body comprising:
a first laterally-tapered end;
a second laterally-tapered end;
an arcuate, non-slippery upper surface extending longitudinally between the first end and the second end;
a lower surface extending longitudinally between the first end and the second end; said lower surface with:
a tunnel portion surface defining a distally-directed, laterally-extending, non-deformable tunnel; and
a skin-engaging portion surface configured for circumferential installation about the waistline of a patient in engagement with the skin of the patient.
